(19) United States
(12) Patent Application Publication
(10) Pub. No.: US 2003/0235574 A1
(43) Pub. Date: Dec. 25, 2003

(54) Enzyme impregnated membranes

(76) Inventors: Rahul Tandon, Grayslake, IL (US); Sujatha Karoor, Lake Bluff, IL (US); Robin G. Pauley, Lake Villa, IL (US); Daniel R. Boggs, Libertyville, IL (US); Rosa H. Yeh, Libertyville, IL (US)

Correspondence Address: BAXTER HEALTHCARE CORPORATION, RENAL DIVISION, 1 BAXTER PARKWAY, DF3-3E, DEERFIELD, IL, 60015, US

(21) Appl. No.: 10/172,657

(22) Filed: Jun. 14, 2002

(51) Int. Cl. A61K 38/46

(52) U.S. Cl. 424/94.6

(57) ABSTRACT

Membranes impregnated with cross-linked enzyme crystals and devices, systems and methods of producing and using same are provided. The present invention includes membranes impregnated with a sufficient amount of a cross-linked enzyme crystal(s), such as membranes impregnated with cross-linked urease crystals. The membrane can been dried in a glycerol solution prior to use.

### BACKGROUND OF THE INVENTION

The present invention relates generally to enzyme impregnated membranes. More specifically, the present invention relates to membranes impregnated with cross-linked enzyme crystals that can be employed in a variety of different applications, such as converting uremic toxins in dialysate during dialysis therapy.

Due to disease or insult or other causes, the renal system can fail. In renal failure of any cause, there are several physiological derangements. The balance of water, electrolytes (e.g., Na, K, Cl, Ca, P, Mg, SO₄ and the like) and the excretion of daily metabolic load of fixed hydrogen ions is no longer possible in renal failure. During renal failure, toxic end products of nitrogen metabolism (e.g., urea, creatinine, uric acid and the like) can accumulate in blood and tissues.

Dialysis processes have been devised for the separation of elements in a solution by diffusion across a semi-permeable membrane (diffusive solute transport) down a concentration gradient. Principally, dialysis comprises two methods: hemodialysis and peritoneal dialysis.

Hemodialysis treatment utilizes the patient's blood to remove waste, toxins, and excess water from the patient. The patient is connected to a hemodialysis machine and the patient's blood is pumped through the machine. For example, needles can be inserted into the patient's veins and arteries to connect the blood flow to and from the hemodialysis machine. Waste, toxins, and excess water are removed from the patient's blood and the blood is infused back into the patient. Hemodialysis treatments last several hours and are generally performed in a treatment center about three or four times per week.

Peritoneal dialysis utilizes a dialysis solution and dialysate, which is infused into a patient's peritoneal cavity. The dialysate contacts the patient's peritoneal membrane in the peritoneal cavity. Waste, toxins, and excess water pass from the patient's bloodstream through the peritoneal membrane and into the dialysate. The transfer of waste, toxins, and water from the bloodstream into the dialysate occurs due to diffusion and osmosis. The spent dialysate is drained from the patient's peritoneal cavity to remove the waste, toxins, and water from the patient.

There are various types of peritoneal dialysis, including continuous ambulatory peritoneal dialysis (“CAPD”) and automated peritoneal dialysis (“APD”). CAPD is a manual dialysis treatment in which the patient connects an implanted catheter to a drain and allows a spent dialysate fluid to drain from the peritoneal cavity. The patient then connects to a bag of fresh dialysate and manually infuses the fresh dialysate through the catheter and into the patient's peritoneal cavity. The patient disconnects the catheter from the fresh dialysate bag and allows the dialysate to dwell within the cavity to transfer waste, toxins, and excess water from the patient's bloodstream to the dialysate solution. After the dwell period, the patient repeats the manual dialysis procedure.

In CAPD, the patient performs several drain, fill, and dwell cycles during the day, for example, about four times per day. Each treatment cycle typically takes about 3-4 hours. Manual peritoneal dialysis performed by the patient requires a great deal of time and effort by the patient. The patient is routinely inconvenienced leaving ample opportunity for therapy enhancements to improve patient quality of life.

Automated peritoneal dialysis is similar to continuous peritoneal dialysis in that the dialysis treatment includes a drain, fill, and dwell cycle. However, a dialysis machine automatically performs 3-4 cycles of peritoneal dialysis treatment, typically overnight while the patient sleeps.

To this end, a dialysis machine is fluidly connected to an implanted catheter. The dialysis machine is also fluidly connected to a source of fresh dialysate, such as a bag of dialysate solution, and to a fluid drain. The dialysis machine pumps spent dialysate from the peritoneal cavity though the catheter to the drain. Then, the dialysis machine pumps fresh dialysate from the dialysate source through the catheter and into the patient's peritoneal cavity. The dialysis machine allows the dialysate to dwell within the cavity to transfer waste, toxins, and excess water from the patient's bloodstream to the dialysate solution. The dialysis machine is computer controlled so that the dialysis treatment occurs automatically when the patient is connected to the dialysis machine, for example, overnight.

Several drain, fill, and dwell cycles will occur during the treatment. Also, a last fill is typically used at the end of the automated dialysis treatment so that the patient can disconnect from the dialysis machine and continue daily functions while dialysate remains in the peritoneal cavity. Automated peritoneal dialysis frees the patient from manually performing the drain, dwell, and fill steps, and can improve the patient's dialysis treatment and quality of life.

In view of recent developments and therapies, the line between traditional peritoneal dialysis and hemodialysis has become blurred. For example, some therapies use components of both therapies.

A recent therapy is regenerative dialysis. In this therapy, a dialysis system is used that includes a cartridge for dialysate regeneration. The cartridge includes a resin bed including zirconium-based resins. The cartridge can also include a layer of an enzyme, such as urease, to convert urea in the dialysate into ammonia and carbon dioxide. Thus, urease is used to remove urea from the dialysate such that the dialysate can be reused.

An example of a cartridge that is used in such a system is manufactured under the name REDY by SORB TECHNOLOGY, Oklahoma City, Okla. This system, however, requires the constant attention of medical personnel. The urease in this type of system is derived from Jack bean meal which is a very impure form of the urease enzyme. In this regard, jack bean meal typically contains lectins and/or other undesirable impurities. Moreover, urease is typically blended with alumina and contained between two layers of alumina within the cartridge. This requires the extensive use of alumina to prevent urease from leaching out of the resin bed due to the high water solubility of urease. This can also require the use of an excessive amount of urease during use to compensate for any potential loss thereof due to its solubility in the dialysate. In the REDY cartridge, the leaching of alumina, urease and/or other impurities from Jack bean meal can be problematic.

A need, therefore, exists to provide improved enzyme impregnated membranes for a variety of suitable applications, such as regeneration of dialysate for reuse during dialysis therapy.

### SUMMARY OF THE INVENTION

The present invention provides membranes impregnated with cross-linked enzyme crystals, devices, systems and methods of producing and using same for a variety of suitable applications including, for example, the removal of uremic toxins from dialysate during dialysis therapy. In this regard, the enzyme impregnated membranes of the present invention can enzymatically convert the uremic toxins into by-products, thus allowing the dialysate to be reused during therapy. This can effectively minimize the amount of dialysate necessary for therapy, thus enhancing therapy and minimizing costs.

In an embodiment, the present invention provides a material including a membrane impregnated with about 3.25 mg/cm² or less of a cross-linked enzyme crystal. Preferably, the membranes are impregnated with cross-linked urease crystals (“urease CLEC”). The membranes impregnated with urease CLECs can be used to remove a therapeutic effective amount of urea from dialysate during therapy allowing the dialysate to be reused.

Applicants have found that by using membranes impregnated with an amount of cross-linked enzyme crystal, such as urease CLEC, less urease can be used than that typically used in sorbent cartridges to remove urea from dialysate. In addition to high enzymatic-activity (about 750 units/mg), it is believed that this can be attributed to the fact that urease CLEC is effectively insoluble in water as compared to the high water solubility of typically used urease materials. In this regard, it is believed that the urease CLEC impregnated within a polymer matrix of the membrane can be better contained in the sorbent cartridge such that excessive amounts thereof are not required to compensate for any potential loss of same during use. Further, the urease CLEC impregnated membranes of the present invention can be used without alumina or the like typically used to minimize leaching of urease and alumina from sorbent cartridges during therapy as previously discussed. Applicants have also found that the enzyme activity of the enzyme impregnated membranes of the present invention remains stable after exposure to gamma-radiation.

In an embodiment, the present invention provides a method of producing a membrane impregnated with cross-linked enzyme crystals, preferably urease CLEC. The method includes preparing a membrane casting solution. The casting solution includes a polymeric base material, such as polyurethane, in a solvent, such as 1-methyl-2-pyrrolidinone (“NMP”), dimethylformamide (“DMF”), the like or combinations thereof. The membrane casting solution can also include a bulking agent, such as zirconium oxide, and an agent, such as polyvinylpyrrolidone (“PVP”) to render the membrane more hydrophilic.

The casting solution is then mixed with a suitable amount of urease CLEC, such that the membrane is impregnated with about 3.25 mg/cm² or less of the urease CLEC and/or other enzyme CLEC. The solution can then be spread on a support material, such as a synthetic mesh material, and immersed into an aqueous media under suitable conditions, thus forming a membrane precipitate. The membrane precipitate is subsequently dried in a glycerol solution, preferably a mixture of glycerol and water at 40:60. Applicants have found that drying the membrane precipitate in a glycerol solution prior to use can effectively preserve enzyme activity.

In an embodiment, the enzyme impregnated membranes of the present invention can be used to detect one or more constituents of a suitable fluid in contact with the membranes. The constituents can include any suitable constituent, such as urea, that is enzymatically reactive with the enzyme, such as urease, of the impregnated membranes.

An advantage of the present invention is to provide improved enzyme impregnated membranes suitable for use in a variety of different applications.

A further advantage of the present invention is to provide improved materials capable of removing uremic toxins from dialysate by converting the uremic toxins into by-products during therapy.

Another advantage of the present invention is to provide improved membranes impregnated with a sufficient amount of cross-linked enzyme crystals.

Yet another advantage of the present invention is to provide improved methods for producing membranes impregnated with cross-linked enzyme crystals.

Still yet another advantage of the present invention is to provide improved membranes impregnated with cross linked urease crystals.

Moreover, an advantage of the present invention is to provide an improved device capable of removing uremic toxins from dialysate during dialysis therapy.

Still, an advantage of the present invention is to provide improved methods and systems for providing dialysis.

Additional features and advantages of the present invention will be described in and apparent from the detailed description of the presently preferred embodiments and the figures.

### BRIEF DESCRIPTION OF THE FIGURES

FIG. 1 is a schematic illustration of a device including a membrane impregnated with a cross-linked enzyme crystal according to an embodiment of the present invention.

FIG. 2 is a schematic illustration of an apparatus including a membrane impregnated with a cross-linked enzyme crystal according to an embodiment of the present invention.

FIG. 3. is a graphical illustration of the results of Experiment No. 1.

### DETAILED DESCRIPTION OF THE PRESENTLY PREFERRED EMBODIMENTS

The present invention provides enzyme impregnated materials suitable for use in a variety of applications including, for example, the removal of uremic toxins from dialysate by converting the uremic toxins into by-products during dialysis therapy and devices, systems and methods of producing and using same. More specifically, the present invention relates to polymeric membranes impregnated with a cross-linked enzyme crystal(s) capable of converting toxins in spent dialysate, thus allowing the dialysate to be reused during therapy. Such enzyme impregnated membranes can also be used in therapeutic, diagnostic and other industrial applications.

It should be appreciated that the present invention can be used in a variety of different dialysis therapies to treat kidney failure. Dialysis therapy as the term or like terms are used throughout the text is meant to include and encompass any and all forms of therapies that provide methods for removing from the patient's blood waste, toxins and/or excess water from the patient. Such therapies include hemodialysis, hemofiltration, hemodiafiltration and peritoneal dialysis including automated peritoneal dialysis, continuous ambulatory peritoneal dialysis and continuous flow peritoneal dialysis. Such therapies can also include, where applicable, both intermittent therapies and continuous therapies used for continuous renal replacement therapy (CRRT). Examples of continuous therapies used in CRRT include slow continuous ultrafiltration (SCUF), continuous venovenous hemofiltration (CVVH), continuous venovenous hemodialysis (CVVHD), continuous venovenous hemodiafiltration (CVVHDF), continuous arteriovenous hemofiltration (CAVH), continuous arteriovenous hemodialysis (CAVHD), continuous arteriovenous hemodiafiltration (CAVHDF), continuous ultrafiltration periodic intermittent hemodialysis or the like.

Further, although the present invention, in an embodiment, can be utilized in methods for providing dialysis therapy to patients having chronic kidney failure or disease, it should be appreciated that the present invention can be used for acute dialysis needs, for example, in an emergency room setting. Lastly, as one of skill in the art appreciates, various forms of dialysis therapy, such as hemofiltration, hemodialysis, hemodiafiltration and peritoneal dialysis may be used in an in center, self/limited care as well as in home settings.

In an embodiment, the present invention includes membranes impregnated with a cross-linked enzyme crystal. The cross-linked enzymes can include any suitable cross-linked enzyme made from a variety of suitable enzymes. In an embodiment, the cross-linked enzymes can include enzymes capable of removing uremic toxins or the like from dialysate, such as, urease, creatinine deiminase, uricase, like enzymes or combinations thereof. In this regard, the enzymes can convert the uremic toxins present in the dialysate into by-products via an enzymatic reaction, thus effectively removing the uremic toxins from the dialysate. For example, urease can enzymatically convert urea into ammonia and carbon dioxide; creatinine deiminase can enzymatically convert creatinine into ammonia and N-methylhydantoin; and uricase can enzymatically convert uric acid in water and oxygen into carbon dioxide, allantoin and hydrogen peroxide. Preferably, the cross-linked enzyme crystal of the present invention includes urease CLEC.

The cross-linked enzyme crystals can be made in any suitable way and are commercially available. For example, the enzyme crystals can be initially prepared, for example, by precipitation from an aqueous solution. Once the crystals are formed, they can then be cross-linked by any suitable cross-linking agent, such as glutaraldehyde, under suitable processing conditions. The cross-linked enzyme crystals can then be further processed, such as by lyophilization.

In an embodiment, the present invention includes a polymeric membrane impregnated with the cross-linked enzyme crystals, preferably urease CLEC, alone or in combination with other cross-linked enzyme crystals. It is believed that the use of urease CLEC impregnated membranes will provide a number of benefits over currently available regenerative dialysis therapy technologies including, for example:

1) Better enzyme containment;

2) Reduced cartridge size resulting in enhanced ease of use by patient;

3) Ease of use during cartridge manufacture; and

4) Increased safety over the existing system (due to better containment of urease in the cartridge).

As previously discussed, the cross-linked enzyme crystals, such as urease CLECs, are effectively insoluble in aqueous solutions, such as dialysate, as compared to urease materials typically used to remove uremic toxins from dialysate. In this regard, it is believed that the urease CLEC impregnated membranes can remain better contained in the cartridge during use without requiring the excessive use of urease to compensate for any potential loss during use and/or the additional use of other materials, such as alumina which is typically used to prevent urease from leaching from the cartridge during use. This can effectively reduce the cost and expense typically associated with using urease materials and/or other like enzyme materials during regenerative dialysis therapy or other suitable applications.

The cross-linked enzyme impregnated membranes of the present invention can be made in a variety of suitable ways. In general, a polymer-based membrane casting solution is first prepared and then mixed with the desired amount and types of cross-linked enzyme crystals. It should be appreciated that the membrane casting solution can be made from any suitable polymer-based materials. Once formed and mixed with the cross-linked enzyme, the membrane casting solution is applied to a support material by, for example, spreading on the support material, and subjected to one or more precipitation and washing sequences to form a composite membrane which can be subsequently dried prior to use.

In an embodiment, the casting solution is composed of a polymeric base material, such as polyurethane or the like, in any suitable solvent including, for example, 1-methyl-2-pyrrolidinone (“NMP”), dimethylformamide (“DMF”), the like or combinations thereof. The casting solution can also include additional other components, such as a bulking agent, a hydrophilic agent (e.g., an agent that can render the membrane more hydrophilic), the like or combinations thereof. In an embodiment, the bulking agent can include zirconium oxide, zirconium phosphate, carbon, the like or combinations thereof. The bulking agent can be added in a sufficient amount to control the porosity of the membrane. The bulking agent can be added in an amount of up to about 80% of the total dry weight of the membrane, preferably about 50% of the total dry weight of the membrane. In an embodiment, the bulking agent and the cross-linked enzyme crystal are added in equal amounts or at least approximately equal amounts.

In an embodiment, the hydrophilic agent is polyvinylpyrrolidone (“PVP”), the like or combinations thereof. The hydrophilic agent can be added in any suitable amount to enhance the hydrophilic nature of the membrane.

The casting solution is then mixed with a suitable amount of a cross-linked enzyme or combinations thereof. In an embodiment, the casting solution is mixed with urease CLEC. As previously discussed, Applicants have found that the amount of cross-linked enzyme, preferably urease CLEC, can be used in reduced amounts. In an embodiment, the cross-linked enzyme is added to the membrane in an amount effective to provide a desired level of enzyme activity. In an embodiment, the membrane is impregnated with about 3.25 mg/cm² or less of the cross-linked enzyme, preferably about 1.5 mg/cm² or less. In this regard, the cross-linked enzyme crystal can amount to about 80% or less of the membrane weight.

The resultant membrane solution is then applied to a support, such as a synthetic mesh material, and immersed into water or other suitable media, such as a mixture of isopropyl alcohol and water, preferably a 50:50 ratio of isopropyl alcohol (“IPA”) to water. A polymer membrane composite material can then be precipitated under suitable conditions. For example, a suitable amount of NMP can be added during water precipitation to control the rate of precipitation. In this regard, the rate of precipitation can be decreased, thus resulting in a more porous polymeric matrix of the membrane.

In an embodiment, the membrane precipitate is dried prior to use. The membrane of the present invention can be dried in any suitable manner, such as air drying, drying with a solvent or the like. Preferably, the membrane precipitate is dried in a glycerol solution composed of glycerol and water. Applicants have found that drying the polymer membrane composite in the glycerol solution prior to use can prevent loss of enzyme activity or at least effectively minimize loss of same during use. It should be appreciated that any suitable amount of glycerol and water can be used to preserve enzyme activity. In an embodiment, the membrane precipitate is dried in a glycerol/water mixture with a 40:60 ratio of glycerol to water. In an embodiment, the glycerol solution contains 40% by weight of glycerol. It should be appreciated that the membrane can be dried with any variety of number and suitable types of fluid mediums including, for example, solvents, solutions, organic solutions including any suitable solvents, such as ethylene glycol, glycerol or other suitable organic solvents, and/or other suitable fluid mediums.

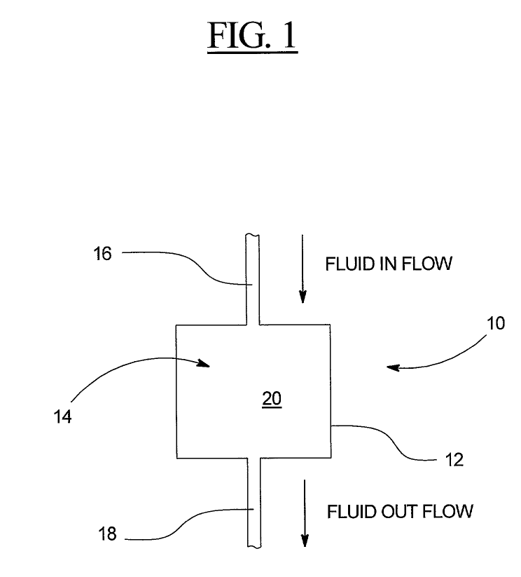

As previously discussed, the cross-linked enzyme impregnated membranes of the present invention can be effectively utilized to remove uremic toxins from dialysate by converting the uremic toxins into enzymatic by-products during dialysis therapy. As applied, the present invention, in an embodiment, can include a device 10 for removing uremic toxins, such as urea, from dialysate or any suitable fluid as shown in FIG. 1. The device 10 can be made in any suitable configuration, such as a sorbent cartridge used during dialysis. In an embodiment, the device 10 includes a body 12 that defines an interior 14 with an inlet 16 and an outlet 18. The interior 14 includes a layer of the cross-linked enzyme impregnated membrane 20, such as an urease CLEC impregnated membrane capable of removing urea from the dialysate as it passes through the device 10 during dialysis therapy. The device can be coupled and used in any suitable system, such as a system for providing dialysis therapy.

It should be appreciated that the device can include one or more suitable resin materials in addition to the cross-linked enzyme impregnated membranes. The additional other resin materials can be used to remove various types of metabolic waste, toxins and/or other organic molecules such as uric acid, creatinine, phosphates, the like or combinations thereof. For example, the device can include a layer of zirconium oxide to remove phosphates. In addition, the device may also include a layer of carbon or activated carbon. In general, carbon can be used to remove creatinine, uric acid, like organic substituents or combinations thereof.

It should be appreciated that the enzyme impregnated membranes of the present invention can be enzymatically reactive to any suitable constituent in solution depending on the type or types of enzymes employed including, for example, glucose oxidase, creatinine deiminase, urease, lactate oxidase, dehydrogenase, phosphatase, such as alkaline phosphotase, sulfatase, such as arylsulfatase, other suitable enzymes and combinations thereof. In this regard, the cross-linked enzyme impregnated membranes of the present invention can be applied in a variety of suitable applications including, for example, therapeutic, diagnostic and/or the like. The membranes can be used in any suitable fluid medium including, for example, aqueous solutions, nonaqueous solutions, dialysate, blood, urine, medical solutions, the like or combinations thereof.

For example, the CLEC impregnated membranes can be used to detect one or more constituents in solution, such as the detection of uremic toxins in dialysate used during dialysis therapy.

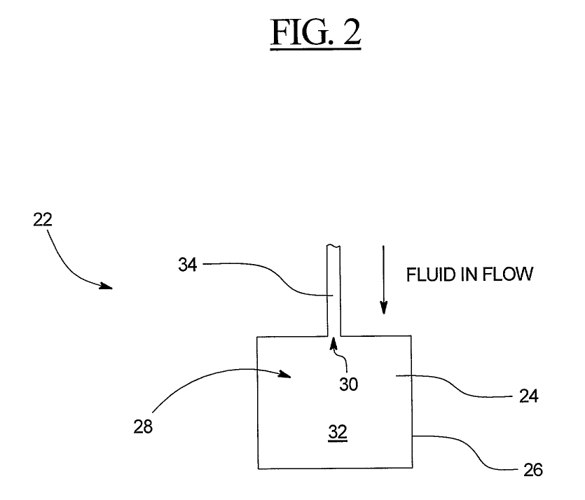

In an embodiment, the present invention includes an apparatus 22 that employs one or more CLEC impregnated membranes to detect the presence and/or amount of one or more constituents in any suitable fluid as shown in FIG. 2. The apparatus 22 of the present invention can include any number and type of suitable components. In an embodiment, the apparatus 22 includes a device 24 with a housing 26 that defines an interior 28 with at least an inlet fluid pathway 30 through which fluid can flow into the interior 28 to contact the CLEC impregnated membrane 32 provided therein. Optionally, the device 24 can include one or more additional fluid pathways (not shown), such as an outlet fluid pathway.

The device 24 can be coupled to a fluid line 34 in any suitable way allowing the fluid to contact the membrane. It should be appreciated that contact made between the fluid and the membrane can be made in such a way that the fluid does not necessarily pass all the way through the thickness of the membrane. In this regard, the enzyme(s) of the CLEC impregnated membrane is capable of enzymatically reacting with any suitable constituent in the fluid for detection purposes. For example, the CLEC impregnated membrane of the present invention is capable of reacting with uremic toxins in dialysate. The by-product(s) of the enzymatic reaction can then be detected by any suitable detection technique including, for example, optical detection, such as colorimetric detection, the like and combinations thereof. The amount of by-product detected can be correlated to the amount of constituent(s) in the fluid.

It should be appreciated that the detection capabilities of the apparatus of the present invention can be carried out in any suitable manner. For example, the device can include or be adaptedly coupled to any suitable type and number of electrical-based components (not shown) for detection purposes. In an embodiment, the device can include or can be adaptedly coupled to opto-electronic circuits (not shown) which can be utilized to convert optical responses, such as colorimetric responses, based on the amount of by-products from the enzymatic reaction into concentration values associated with the detectable constituents, such as urea, in the solution.

The detection capabilities of the apparatus of the present invention can be used to monitor any suitable fluid process. In an embodiment, the present invention can be utilized to monitor the amount of uremic toxins removed from a patient during dialysis therapy. This can provide day-to-day trends of total removals of uremic toxins and thus be used to evaluate clearance levels during dialysis therapy such that the therapy can be effectively monitored and/or controlled.

By way of example and not limitation, the experiments below set forth further embodiments and analysis of the invention.

Experiment No. 1

In this experiment, the effects of post treatment on enzyme activity of the urease CLEC were evaluated. The urease CLEC impregnated membranes were made by forming a membrane casting solution with polyurethane in a specific type of solvent; adding urease CLEC to the casting solution; precipitating an impregnated membrane composite material in a suitable media; and optionally processing the composite material by drying prior to use. Specific processing conditions, such as the type of membrane casting solvent, amount of urease CLEC, precipitation bath media and post treatment conditions, for each of the test impregnated membranes are identified in Table 1 below:

TABLE 1

| | Solvent | Precipitation | Urease Amt. | Post |
|---|---|---|---|---|
| Membrane | System | Bath | (mg) | Treatment |
| 1 | DMF | 50/50 IPA & Water | 9.33 | Wet, never |
| | | | | dried |
| 2 | DMF | 50/50 IPA & Water | 14.58 | 40% glycerol |
| | | | | dried |
| 3 | DMF | 50/50 IPA & Water | 15.30 | Wet, never |
| | | | | dried |

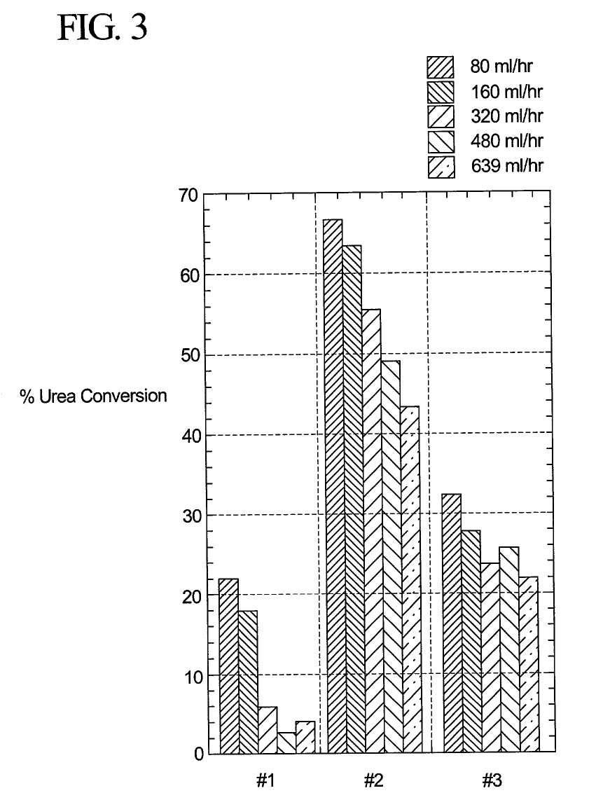

The urease activity was tested for each of the test impregnated membranes identified in Table 1. In this regard, the urea conversion rates were conventionally measured for each of the membranes based on the conversion of urea in solution into carbon dioxide and ammonia at varying flow rates. As shown in FIG. 3, the urease activity for the test impregnated membrane No. 2 exhibited considerable retention of enzyme activity subsequent to drying in glycerol.

Experiment No. 2

In this experiment, two groups of test impregnated membranes were made, namely Groups A and B. Group A membranes (e.g., A1-A2) were made from polyurethane in a NMP solvent. Group B membranes (e.g., B1-B2) were made from polyurethane in a DMF solvent. The impregnated membranes were about 1 inch in diameter. Specific processing conditions are identified below in Table 2:

TABLE 2

| | | | | | | % Urea |
|---|---|---|---|---|---|---|
| | | Solution | | | | Conver- |
| | | in Pre- | | | | sion |
| | | cipita- | Urease | Post- | | @ |
| Membrane | | tion | Amount | Treat- | Gamma- | 320 |
| No. | Solvent | Bath | (mg) | ment | exposure | ml.hr |
| A1 | NMP | Water | 14.81 | 40% | None | 90.8 |
| | | | | glycerol | | |
| | | | | dried | | |
| A2 | NMP | Water | 15.74 | 40% | Yes | 85.0 |
| | | | | glycerol | (14.8 kGy) | |
| | | | | dried | | |
| B1 | DMF | 50/50 | 15.01 | 40% | Yes | 33 |
| | | IPA & | | glycerol | (39.7 kGy) | |
| | | Water | | dried | | |
| B2 | DMF | 50/50 | 14.87 | 40% | None | 35.3 |
| | | IPA & | | glycerol | | |
| | | Water | | dried | | |

Once formed, a membrane from each group was exposed to gamma radiation at certain dosages as indicated in Table 2. The other membrane in each group was used as a control with no exposure to gamma-radiation. The urease activity of each of the membranes was then tested with a urea test solution to determine % urea conversion as discussed in Experiment No. 1. The urease activities of the membranes at a flow rate of the test solution of 320ml/hr are indicated in Table 2. The results from Experiment No. 2 indicate that the urease CLEC impregnated polyurethane membranes made in a NMP solvent displayed greater retention of urease activity as compared to the membranes made in a DMF solvent. Further, the results of Experiment No. 2 demonstrate that the exposure to gamma-radiation has negligible, if any, effect on enzyme activity as compared to impregnated membranes without such exposure.

Experiment No. 3

In this experiment, the effect of the type of urease source on urease activity was tested. Three groups of membranes were made for this experiment. The membranes of group C (e.g., C1-C8) were impregnated with varying amounts of Jack Bean Meal, a commercially available urease source (Sigma Cat #J0125); the membranes of group D (e.g., D1-D4) were impregnated with varying amounts of urease CLEC; and the membranes of group E (e.g., E1-E4) were impregnated with varying amounts of a commercially available source of pure urease enzyme (Roche Lot #85768329). The membranes had a diameter of about 1 inch and a thickness of about 200 microns. Specific membrane processing conditions are listed below as indicated in Table 3:

TABLE 3

| | | | | | | % |
|---|---|---|---|---|---|---|
| | | | | | | Urea |
| | | | Urease | Post | Gamma | Con- |
| | Solvent | Urease | Amt. | Treat- | Sterili- | ver- |
| Membrane | System | Type | (mg) | ment | zation | sion |
| C1 | NMP | Jack Bean | about 34 | 40% | No | 2.90 |
| | | Meal | | glycerol | | |
| | | | | dried | | |
| C2 | NMP | Jack Bean | about 33 | 40% | Yes | 1.00 |
| | | Meal | | glycerol | | |
| | | | | dried | | |
| C3 | NMP | Jack Bean | about 33 | 40% | No | 6.95 |
| | | Meal | | glycerol | | |
| | | | | dried | | |
| C4 | NMP | Jack Bean | about 34 | 40% | Yes | 2.90 |
| | | Meal | | glycerol | | |
| | | | | dried | | |
| C5 | NMP | Jack Bean | about 71 | 40% | No | 17.70 |
| | | Meal | | glycerol | | |
| | | | | dried | | |
| C6 | NMP | Jack Bean | about 70 | 40% | Yes | 3.40 |
| | | Meal | | glycerol | | |
| | | | | dried | | |
| C7 | NMP | Jack Bean | about 71 | 40% | No | 30.80 |
| | | Meal | | glycerol | | |
| | | | | dried | | |
| C8 | NMP | Jack Bean | about 70 | 40% | Yes | 6.40 |
| | | Meal | | glycerol | | |
| | | | | dried | | |
| D1 | NMP | Urease | 15.05 | 40% | No | 70.10 |
| | | CLEC | | glycerol | | |
| | | | | dried | | |
| D2 | NMP | Urease | 15.03 | 40% | Yes | 74.30 |
| | | CLEC | | glycerol | | |
| | | | | dried | | |
| D3 | NMP | Urease | 15.25 | 40% | No | 70.05 |
| | | CLEC | | glycerol | | |
| | | | | dried | | |
| D4 | NMP | Urease | 15.85 | 40% | Yes | 79.00 |
| | | CLEC | | glycerol | | |
| | | | | dried | | |
| E1 | NMP | Pure urease | 25.50 | 40% | No | 10.06 |
| | | enzyme | | glycerol | | |
| | | | | dried | | |
| E2 | NMP | Pure urease | 25.39 | 40% | Yes | 1.60 |
| | | enzyme | | glycerol | | |
| | | | | dried | | |
| E3 | NMP | Pure urease | 25.20 | 40% | No | 14.81 |
| | | enzyme | | glycerol | | |
| | | | | dried | | |
| E4 | NMP | Pure urease | 25.98 | 40% | Yes | 10.70 |
| | | enzyme | | glycerol | | |
| | | | | dried | | |

The urease activity as measured by % urease conversion based on conversion of urea in a test solution was then evaluated for each of the membranes listed in Table 3. The results of Table 3 indicate that the membranes impregnated with urease CLEC displayed a higher activity at lower amounts of urease as compared to membranes impregnated with typically used urease materials, e.g., Jack Bean Meal and pure urease enzyme.

Experiment No. 4

In this experiment, the effects of the size of the membrane on urease activity were tested. Two membranes were impregnated with urease CLEC similar to how the test membranes were made as discussed in the other experiments. However, the membranes were larger in size as compared to the membranes of the previous experiments. In particular, the membranes of this experiment were about 3.5 inches in diameter with a thickness of about 200 microns. The membranes also contained a larger amount of urease CLEC as indicated in Table 4:

TABLE 4

| | | | | % Urea |
|---|---|---|---|---|
| | Solvent | Urease Amt. | Post | Conversion at |
| Membrane | System | (mg) | Treatment | 100 ml/min |
| 4A | NMP | 181.38 | 40% Glycerol | 72.0 |
| | | | dried | |
| 4B | NMP | 181.57 | 40% Glycerol | 63.1 |
| | | | dried | |

The urease activity was tested by passing a test solution of 20 mg/dl of urea through the membrane. The % urea conversion was then calculated as further indicated in Table 4. The results indicate that the CLEC impregnated membranes of the present invention can be scaled to any suitable size depending on its desired use.

It should be understood that various changes and modifications to the presently preferred embodiments described herein will be apparent to those skilled in the art. Such changes and modifications can be made without departing from the spirit and scope of the present invention and without diminishing its intended advantages. It is therefore intended that such changes and modifications be covered by the appended claims.

The invention is claimed as follows:

1. A method of producing a membrane, the method comprising the steps of:
preparing a casting solution composed of a polymeric base material in a solvent;
adding a sufficient amount of a cross-linked enzyme crystal to the casting solution;
applying the membrane casting solution to a support material;
immersing the membrane casting solution and the support material in an aqueous media;
forming a membrane composite material; and
drying the membrane composite material with a fluid medium.

2. The method of claim 1 wherein the polymeric base material is composed of a suitable polymer including polyurethane.

3. The method of claim 1 wherein the cross-linked enzyme crystal includes an enzyme selected from the group consisting of urease, creatinine deiminase, uricase, glucose oxidase, lactate oxidase, dehydrogenase, phosphatase, alkaline phosphotase, sulfatase, arylsulfatase and combinations thereof.

4. The method of claim 1 wherein the solvent is selected from the group consisting of 1-methyl-2-pyrrolidinone, dimethylformamide and combinations thereof.

5. The method of claim 1 wherein the membrane is impregnated with about 3.25 mg/cm² or less of the cross-linked enzyme crystal.

6. The method of claim 1 wherein the fluid medium includes glycerol.

7. The method of claim 1 the fluid medium includes a glycerol solution having 40% by weight of glycerol.

8. A method of producing an urease impregnated membrane, the method comprising the steps of:
forming a membrane casting solution including polyurethane and a bulking agent in a solvent;
adding the urease CLEC to the membrane casting solution;
processing the membrane casting solution in an aqueous media; and
forming a membrane precipitate impregnated with about 3.25 mg/cm² of the urease CLEC.

9. The method of claim 8 wherein the membrane precipitate is impregnated with about 1.5 mg/cm² or less of the urease CLEC.

10. The method of claim 8 wherein the bulking agent is selected from the group consisting of zirconium oxide, zirconium phosphate, carbon and combinations thereof.

11. The method of claim 8 wherein the bulking agent and the urease CLEC are added to the casting solution in an equal amount.

12. The method of claim 8 further comprising the step of drying the membrane precipitate with a glycerol solution.

13. The method of claim 12 wherein the glycerol solution includes glycerol and water.

14. The method of claim 13 wherein a ratio of glycerol to water is 40:60.

15. A material capable of removing uremic toxins from dialysate during dialysis therapy, the material comprising a membrane impregnated with about 3.25 mg/cm² or less of a cross-linked enzyme crystal wherein the membrane has been dried with a fluid medium.

16. The material of claim 15 wherein the cross-linked enzyme crystal includes an enzyme selected from the group consisting of urease, creatinine deiminase, uricase, glucose oxidase, lactate oxidase, dehydrogenase, phosphatase, alkaline phosphotase, sulfatase, arylsulfatase and combinations thereof.

17. The material of claim 15 wherein the membrane includes a bulking agent selected from the group consisting of zirconium oxide, zirconium phosphate, carbon and combinations thereof.

18. The material of claim 17 wherein the fluid medium includes glycerol.

19. The material of claim 15 wherein the membrane is impregnated with about 3.25 mg/cm² or less of an urease CLEC.

20. The material of claim 15 wherein the membrane is impregnated with about 1.5 mg/cm² or less of an urease CLEC.

21. A device for removing uremic toxins from a dialysate used during dialysis therapy, the device comprising:
a body defining an interior with an inlet and an outlet, the interior containing a layer of a membrane impregnated with about 3.25 mg/cm² or less of a cross-linked enzyme crystal wherein the membrane has been dried with a fluid medium.

22. The device of claim 21 wherein the cross-linked enzyme crystal includes an enzyme selected from the group consisting of urease, creatinine deiminase, uricase, glucose oxidase, lactate oxidase, dehydrogenase, phosphatase, alkaline phosphotase, sulfatase, arylsulfatase and combinations thereof

23. The device of claim 21 wherein the membrane is impregnated with about 1.5 mg/cm² or less of an urease CLEC.

24. The device of claim 21 wherein the fluid medium includes glycerol.

25. The device of claim 21 wherein the membrane impregnated with a cross-linked urease crystal can be contained in the device without alumina.

26. An apparatus for detecting one or more constituents in a fluid, the apparatus comprising:
a device including a membrane impregnated with a cross-linked enzyme crystal wherein the membrane has been dried with a fluid medium; and
a fluid line coupled to the device through which the fluid can contact the membrane allowing detection of one or more constituents enzymatically reactive with the cross-linked enzyme crystal of the membrane.

27. The apparatus of claim 26 wherein the membrane is impregnated with about 3.25 mg/cm² or less of the cross-linked enzyme crystal.

28. The apparatus of claim 26 wherein the cross-linked enzyme crystal includes an enzyme selected from the group consisting of urease, creatinine deiminase, uricase, glucose oxidase, lactate oxidase, dehydrogenase, phosphatase, alkaline phosphotase, sulfatase, arylsulfatase and combinations thereof.

29. The apparatus of claim 26 wherein the apparatus can optically detect one or more of the enzymatically reactive constituents of the fluid.

30. The apparatus of claim 26 wherein the fluid is dialysate containing uremic toxins removed during dialysis therapy.

31. The apparatus of claim 26 wherein the fluid medium includes glycerol.

32. A system for providing dialysis therapy, the system comprising a device capable of removing uremic toxins from dialysate, the device including a body defining an interior with an inlet and an outlet, the interior containing a layer of a membrane impregnated with about 3.25 mg/cm² or less of a cross-linked enzyme crystal wherein the membrane has been dried with a fluid medium.

33. The system of claim 32 wherein the cross-linked enzyme crystal includes an enzyme selected from the group consisting of urease, creatinine deiminase, uricase, glucose oxidase, lactate oxidase, dehydrogenase, phosphatase, alkaline phosphotase, sulfatase, arylsulfatase and combinations thereof.

34. The system of claim 32 wherein the membrane is impregnated with about 1.5 mg/cm² or less of an urease CLEC.

35. The system of claim 32 wherein the membrane can be contained in the device without alumina.

36. The system of claim 32 wherein the fluid medium includes glycerol.

37. A method of providing dialysis therapy, the method comprising the steps of:
passing a dialysis fluid through a device including a layer of a membrane impregnated with about 3.25 mg/cm² or less of a cross-linked enzyme crystal wherein the membrane has been dried with a glycerol solution; and
removing a therapeutically effective amount of uremic toxins from the dialysis fluid.

38. The method of claim 37 wherein the cross-linked enzyme crystal includes an enzyme selected from the group consisting of urease, creatinine deiminase, uricase, glucose oxidase, lactate oxidase, dehydrogenase, phosphatase, alkaline phosphotase, sulfatase, arylsulfatase and combinations thereof.

39. The method of claim 37 wherein the membrane is impregnated with about 1.5 mg/cm² or less of an urease CLEC.
